(19) United States
(12) Patent Application Publication
(10) Pub. No.: US 2015/0141900 A1
(43) Pub. Date: May 21, 2015

(54) Method and Device for Targeted Delivery of Fluid Therapeutics

(71) Applicant: The Johns Hopkins University, Baltimore, MD (US)

(72) Inventors: Anthony N. Kalloo, Baltimore, MD (US); Mouen A. Khashab, Towson, MD (US)

(21) Appl. No.: 14/358,426

(22) PCT Filed: Nov. 30, 2012
(86) PCT No.: PCT/US2012/067376

§ 371 Date: May 15, 2014

Related U.S. Application Data
(60) Provisional application No. 61/565,643, filed on Dec. 1, 2011.

(51) Int. Cl. A61M 31/00; A61M 25/10; A61M 25/00

(52) U.S. Cl. CPC A61M 31/002; A61M 25/0043; A61M 25/0074; A61M 25/0082; A61M 25/10; A61M 2025/0057; A61M 2025/1052
USPC 604/14

(57) ABSTRACT

The present invention utilizes a catheter device for targeted delivery of therapeutic molecules in fluids to body cavities. The invention provides a device and method for targeted delivery of fluids to tissue cavities via deployment of a fluid soaked sponge from the catheter to contact surfaces within the body cavity.

### BACKGROUND OF THE INVENTION

1. Field of the Invention

The present invention relates generally to medical devices and more specifically to a device and method for targeted delivery of fluid therapeutics to a subject.

2. Background Information

A stricture is an abnormally narrowed segment of an otherwise patent biological tube or conduit, such as the gastrointestinal tract, genito-urinary tract, pulmonary system, vascular system, or other systems in the body. Strictures may occur at various places within these systems in the body, such as in or near a blood vessel, the bronchial tree, the colon, a gastrointestinal body structure, the pancreaticobiliary structure, a genital body structure, a kidney, a post-operative stricture, a pulmonary body structure, the rectum, or the sphincter, or a urethral body structure. The degree of narrowing, the length, and the significance of the stricture may differ greatly between particular strictures, and is responsive to the nature of the conduit which is subject to the stricture. Various etiological factors might be responsible for the development or exacerbation of any particular stricture; these may include, for example, infection, inflammation, trauma (whether external, internal, or iatrogenic or other surgical trauma), or cancer. One or more of these factors causes the lumen of the affected conduit to narrow, that is, to stricture, with consequential obstruction of the lumen and compromise of the function of the conduit.

Other pathologies that are typically associate with physiological tracts present in the body include lesions and ulcers. Lesions and ulcers may be found in any number of the body structures described above.

Treatment of strictures, ulcers and lesions is aimed at restoration of intraluminal patency and physiological function. Because of the presence of abnormal or diseased tissue at the stricture, surgical treatment by endoscopic or by open surgical techniques often poses extra difficulties. Moreover, because the tissue at the stricture or lesion is already diseased or damaged, it often generates further scarring and fibrosis when it heals after surgery, which potentially leads to recurrence of the stricture.

Accordingly, it would be advantageous to provide a method and device for treatment of strictures and lesions, which promote healing of existing tissue with minimal damage to existing tissue, such as by targeted delivery of fluid therapeutics to the damaged tissue site.

### SUMMARY OF THE INVENTION

The present invention provides a catheter device for targeted delivery of fluids to tissue cavities for treatment of diseases and disorders, such as strictures, lesions and ulcers.

Accordingly, in one aspect, the present invention provides a catheter device for targeted delivery of a fluid. The device includes: a) a catheter defining at least one longitudinal lumen extending from a distal end of the catheter to a proximal end of the catheter; and b) a deployable sponge being disposed within the lumen of the catheter. In an un-deployed state, the sponge is compressed within the walls of the lumen and transitions to an expanded state when deployed by advancing the sponge through a hole in the distal end of the catheter to deploy the sponge. Expansion of the sponge facilitates contact of the sponge with a tissue surface. The sponge is in fluid communication with the proximal end of the catheter via a lumen which allows flow of a fluid from the proximal end of the catheter to the sponge such that the fluid is delivered to the tissue upon contact of the deployed sponge with tissue.

In another aspect, the present invention provides a method for targeted delivery of a therapeutic fluid, preferably a medication fluid, to a tissue. The method includes: a) advancing a catheter device of the invention into a tissue cavity of a subject; b) providing a fluid to the sponge via a lumen, c) deploying the sponge through the hole in the distal end of the catheter thereby allowing contact of the sponge with a surface of the tissue cavity, wherein contact of the sponge with the surface of the tissue cavity allows delivery of the fluid to the surface; and d) retracting the sponge and catheter device from the lumen.

In another aspect, the present invention provides a method for long-term delivery of a therapeutic fluid, preferably a medication fluid, to a tissue. The method includes: a) advancing a catheter device of the invention into a tissue cavity of a subject; b) providing a fluid to the sponge via a lumen, c) deploying the sponge through the hole in the distal end of the catheter so it comes into contact with a surface of the tissue cavity; and d) releasing the sponge into the cavity, wherein contact of the sponge with the surface of the tissue cavity allows delivery of the fluid to the surface.

In a particularly preferred embodiment of this aspect of the invention, the lumen is a body space which is subjected to peristaltic or catastalic motion; e.g.,., the gastrointenstinal tract, urethra or bladder. The squeezing force applied to the sponge by the peristaltic motion causes release of the fluid contained therein. In a most preferred embodiment of this aspect of the invention, the sponge is bioresorbable.

In another aspect, the present invention provides a method of treating a disease or condition of a subject accompanying the occurrence of a stricture. The method including delivering a therapeutic molecule, preferably contained in a medication fluid, to a surface of a tissue cavity of the subject using the device of the present invention. In various embodiments, the disease or disorder is disorder is interstitial cystitis, a recalcitrant structure, a lesion, or an ulcer.

### BRIEF DESCRIPTION OF THE DRAWINGS

FIG. 1 is a longitudinal cross-sectional diagram showing a catheter device having proximal (A) and distal (B) ends along with central lumen (50) in one embodiment of the invention.

FIG. 2 is a longitudinal cross-sectional diagram showing a catheter having a distally (B) disposed sponge (20) in one embodiment of the invention.

FIG. 3 is a longitudinal cross-sectional diagram showing a catheter having a distally (B) disposed sponge (20) in one embodiment of the invention.

FIG. 4 is a longitudinal cross-sectional diagram showing a catheter having a distally (B) disposed sponge (20) in one embodiment of the invention.

FIG. 5 is a radial cross-sectional diagram showing a balloon (20) in an un-deployed (A) and a deployed (B) configuration in embodiments of the invention.

FIG. 6 is a radial cross-sectional diagram showing a balloon (20) in an un-deployed (A) and a deployed (B) configuration in embodiments of the invention.

FIG. 7 is a longitudinal cross-sectional diagram showing a catheter device having a distally (B) disposed inflation balloon (60) in one embodiment of the invention.

### DETAILED DESCRIPTION OF THE INVENTION

The present invention provides a catheter device and method for targeted delivery of a fluid, such as a therapeutic or medicated fluid, to a tissue surface of a body cavity. As discussed herein, the device utilizes a deployable sponge, which when deployed from the device to contact a tissue surface within a body cavity, the sponge expands to contact the surrounding tissue surfaces thereby delivering any therapeutic molecule flushable from the surface of a sponge on contact with a fluid or contained in a fluid imbibed by the sponge to the body cavity. For ease of reference, the fluid to be passed through the sponge according to the invention shall be hereinafter referred to the “fluid,” unless context otherwise requires, and shall be understood to mean a fluid into which a therapeutic molecule is suspended or dissolved, whether before passage through the sponge or by release of the molecule from the sponge. In a particular preferred embodiment of the invention, the fluid is released from the sponge to contact only discrete surfaces of the tissue surface of the body cavity.

The device particularly provides means to allow the application of topical fluid therapy during endoscopy and/or laparoscopy procedures. The device of the present invention is feasible to manufacture and the method of its use allows for effective, efficacious, and safe therapy of certain disorders, such as recalcitrant strictures, lesions and ulcers of tissue cavities.

Before the present device and method are described, it is to be understood that this invention is not limited to particular device and methodology described. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The device of the present invention generally includes: a) a catheter defining at least one longitudinal lumen extending from a distal end of the catheter to a proximal end of the catheter; and b) a deployable sponge being disposed within the lumen of the catheter. In an un-deployed state, the sponge is compressed within the walls of the lumen and transitions to an expanded state when deployed by advancing the sponge through a hole in the distal end of the catheter to deploy the sponge.

Expansion of the sponge facilitates contact of the sponge with a tissue surface of a body cavity. The sponge is in fluid communication with the proximal end of the catheter via a lumen which allows for flow of a fluid from the proximal end of the catheter to the sponge. As such, the fluid is delivered through the sponge (whereupon any therapeutic molecule relesably attached to the sponge may become suspended or dissolved in the fluid) to the body cavity upon contact of the deployed sponge with the tissue surface. The fluid itself may, alternatively or additionally, contain a therapeutic molecule.

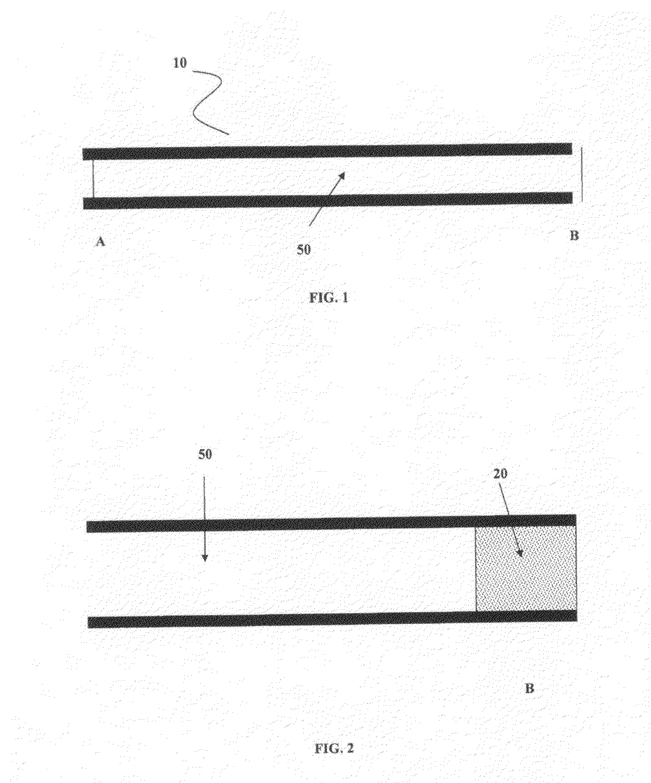

By way of illustration, FIG. 1 generally depicts a longitudinal cross-section of a device (10) of the present invention having proximal (A) and distal (B) ends. The device further includes at least one lumen (50) extending between the proximal and distal ends. In preferred embodiments of the invention, device (10) is deployable within an invasive scope, such as an endoscope or laparascope having at least one lumen therethrough.

FIG. 2 is a longitudinal cross-section of the distal (B) end of the device. Disposed with the distal lumen of the device, a sponge (20) is shown in an un-deployed state. When un-deployed, the sponge (20) is compressed within the lumen (50) and is fully within the lumen (50).

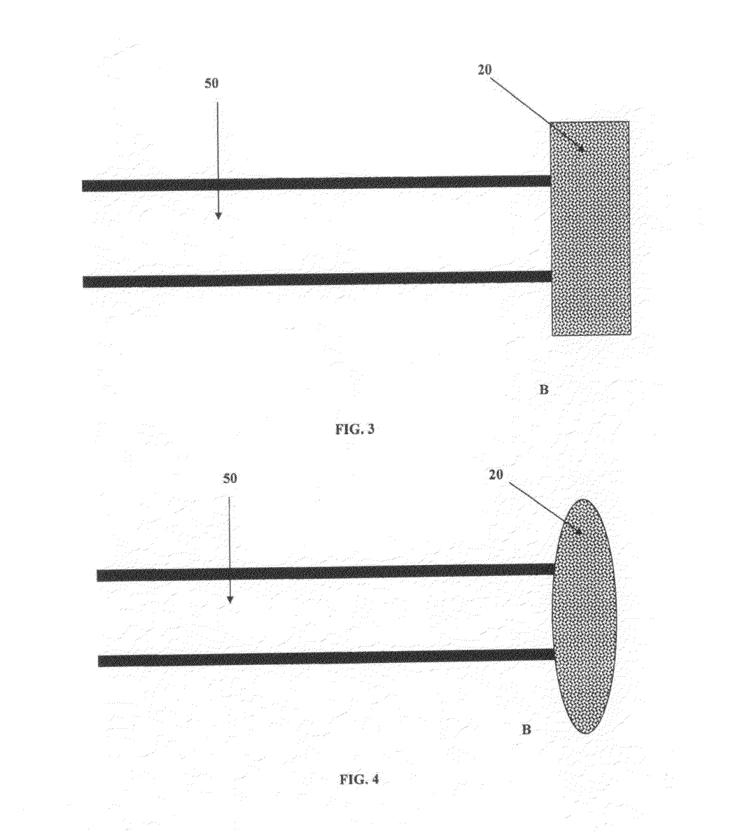

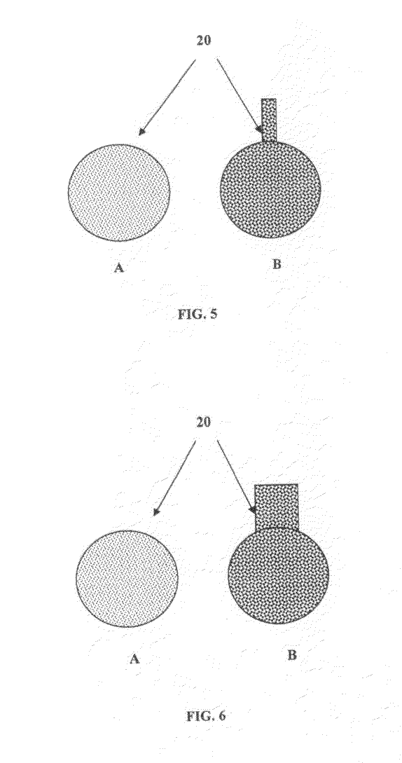

FIGS. 3 and 4 are longitudinal cross-sections of the distal ends (B) of devices of the present invention. In FIGS. 3 and 4, sponge (20) is shown in the deployed state in which the sponge (20) is advanced out of the lumen (50) (for example, by deployment of a pusher structure, such as an axially disposed wire, pusher rod or catheter, through lumen (50) to move sponge (20) distally and, in certain embodiments, to retract it proximally following treatment). On deployment from lumen (50), sponge (20) is allowed to expand within a tissue cavity, for example.

Accordingly, the device has a first configuration wherein the sponge (20) is un-deployed and compressed within the lumen (50). Additionally, the device has a second configuration wherein the sponge (20) is deployed out of the distal end (B) of the lumen (50) and allowed to expand to contact surrounding tissue surfaces. Further, the device optionally has a third configuration wherein the sponge (20) is released from the pusher structure into the tissue cavity.

In the deployed state, contact between the sponge (20) and surrounding tissue facilitates delivery of a fluid supplied to the sponge (20) to be delivered in a discrete and target fashion to the contacted tissue. The fluid may be supplied to the sponge (20) by applying the fluid to the proximal end of the device and allowing the fluid to flow along a lumen in fluid communication with the distally disposed sponge (20). The fluid is absorbed by the sponge (20) and wicked or otherwise released to the tissue. Use of a fluid resistant material (such as the non-absorbent polymers described further herein) to form the sponge assists in ensuring that the therapeutic fluid is released to a discrete and targeted tissue surface.

Accordingly, in practice, the device (10) is advanced into a tissue surface of a body cavity. The sponge is deployed through a hole in the distal end of the catheter thereby allowing contact of the sponge (20) with a surface of the tissue cavity. Contact of the sponge (20) with the surface of the tissue cavity allows delivery of fluid imbibed by the sponge (20) to be delivered to the surface of the tissue. Delivery may be aided by peristaltic or catastaltic motion along the tissue cavity walls, if present; e.g., as in treatment of the gastrointestinal tract.

The sponge may be retracted into lumen (50) of the catheter and withdrawn from the body following contact with the tissue, or released from the catheter to remain in the body following treatment. In the latter instance, the sponge is most preferably made of a bioresorbable material.

As used herein, the term “ sponge”, is intended to refer to a material that is expandable from a first compressed state to a second state having a greater size and volume. In this respect, “deployable” means that the sponge which is axially compressed in line for delivery though the catheter lumen expands upon exit from the lumen. The term “expandable”, as used herein, means the ability of the device to increase in volume.

In general, sponges have the ability to releasably absorb fluid. Additionally, sponges may act to wick fluid along a flow path from a source to a sink. A number of known materials are suitable for use as a sponge in the present invention. The sponge may be composed of a variety of biocompatible materials so long as the materials are substantially non-degradable and exhibit sponge like properties, for example the ability to expand and contract. Biocompatible, non-degradable sponge materials suitable for implantation include polyethylene glycol (PEG) based hydrogels, such as those described in U.S. Patent Application Publication No. 2009/0238815, which is incorporated herein by reference.

Other suitable materials include cross-linked chitosan sponges. Chitosan sponges may be prepared by freeze-drying of high and low molecular weight (e.g., 1.25% and 2.5% (w/w)) chitosan solutions, using a cross-linking agent, such as glutaraldehyde. The hardness and compressibility of the sponges is typically a function of the cross linking agent concentration and volume where an exemplary concentration is about 5%.

Additional suitable sponge materials include, but are not limited to, collagen materials, such as those used in tissue engineering applications (e.g., bone growth scaffolds), silicones, polytetrafluoroethylene (PTFE), polyether ether ketone (PEEK), open cell polyurethane foams and polypropylene.

If a therapeutic molecule is to be reversibly bound to the surface of the sponge, well-known options for attachment include adsorption via mainly hydrophobic interaction (generally between a hydrophobic termini on the sponge material and the hydrophobic terminal of, say, amphiphilic molecules or copolymers), electrostatic interactions, covalent modifications of the sponge surface (e.g., with grafted polymer chains, biotin-avidin complexes and the like). Those of ordinary skill in the art will be familiar with the binding techniques suitable to a particular molecule and the sponge substrate.

In various embodiments, the sponge material is capable of expanding in volume upon decompression. As is known in the art, the increase in volume will be dependent upon the properties of the material used, but typically is capable of increasing in volume by a factor of at least 2, 3, 4, 5, 6, 7, 8, 9, or even 10 when fully expanded.

As illustrated in FIGS. 3 to 6, the sponge (20) of the device may be any geometric shape the facilitates contact of the sponge (20) with the tissue. FIG. 3 shows a cross-section of a deployed sponge (20) having a cylindrical shape. FIG. 4 shows a cross-section of a deployed sponge (20) having an elliptical or circular shape.

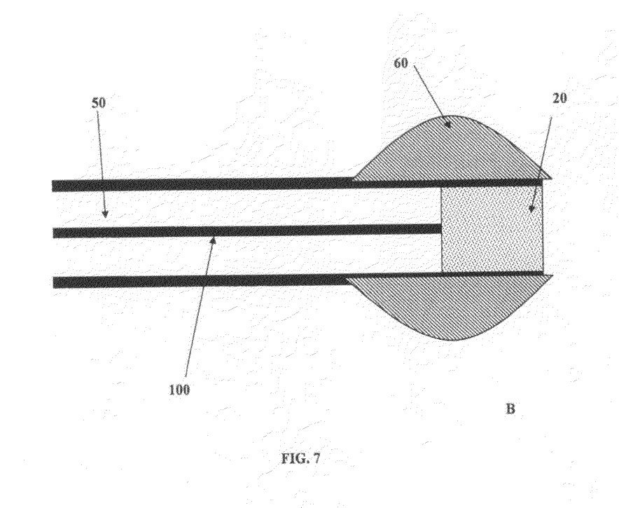

One of skill in the art would appreciate that the shape of the sponge (20) may be configured such that, the fluid may be delivered controllably and discretely to specific areas with a tissue cavity. For example, FIGS. 5 and 6 illustrate embodiments in which the sponge (20) is configured to contact tissue on one side of the device in the expanded or deployed (B) configuration. To achieve similar selective delivery, regions of the sponge (20) may be selectively coated or may incorporate a fluid impervious material the allows migration of fluid only through selective regions of the sponge (20). Such non-adsorptive polymers include those that are widely used in medical devices as described in U.S. Pat. No. 5,169,720 and U.S. Pat. No. 5,039,458, which are incorporated herein by reference. Other suitable coatings include hydrophilic coatings that are employed on surgical devices that work by creating a water barrier as described in U.S. Pat. No. 6,238,799 and U.S. Pat. No. 6,866,936, which are incorporated herein by reference.

With reference to FIG. 7, in one embodiment, the present invention provides a device configured with an inflation balloon (60) disposed at the distal end of the device; e.g., a balloon catheter. The balloon (60) may be disposed at any point along the catheter shaft. In an exemplary embodiment, the balloon (60) is disposed at the distal end of the shaft. In various embodiments, the balloon (60) includes a wall having proximal and distal portions and having interior and exterior surfaces, the interior surface of the balloon wall being secured to the shaft in a fluid-tight manner. To facilitate inflation of the balloon (60) the shaft may include an inflation lumen in fluid communication with the balloon whereby fluid or gas can be infused and withdrawn to inflate and to deflate the balloon. Fluid or gas may be infused or withdrawn through an accessory port in fluid communication with the inflation lumen which may be adapted to couple with a syringe, external pump, or the like.

The embodiment of FIG. 7 further depicts a device including a guidewire or stylus (100), however, it is envisioned that this feature may be included on any embodiment of the present invention discussed herein. In various embodiments the guidewire or stylus (100) is included to facilitate advancement of the sponge (20) distally to deploy the sponge (20) from the catheter lumen at a desired site and subsequently retrieve the sponge (20). In some embodiments the guidewire or stylus (100) remains coupled to the sponge (20) after deployment of the sponge (20) to aide in retrieval of the sponge as well as to allow repositioning of the sponge (20) after deployment without the need for fully recovering the sponge (20).

One skilled in the art would understand that the device may include any number of balloons (60) or other expandable elements disposed along the elongated catheter shaft. For example, 1, 2, 3, 4 or more balloons (60) or other elements may be disposed along the shaft.

The balloon may be made of a compliant material which resiliently deforms under radial pressure. Examples of suitable compliant materials are generally known in the art and include materials such as, but not limited to polyethylene (PE), polyurethane (PU), nylon, silicone, low density polyethylene (LDPE), polyether block amides (PEBAX), and the like. The balloon may also be made of a semi- or non-compliant material. For example, the balloon may include an inelastic fiber layer and/or sleeve. The inelastic fibers are of high-strength and typically made of a high-strength polymeric material. Examples of suitable materials are generally known in the art and include materials such as, but not limited to Kevlar®, Vectran®, Spectra®, Dacron®, Dyneema®, Terlon® (PBT), Zylon® (PBO), polyimides (PIM), other ultra high molecular weight polyethylene (UHMWPE), aramids, and the like. In various embodiments, especially those utilizing a balloon (60), the device may further include a radiopaque material to allow for detection of the position of the device. A number of radiopaque materials and coatings are well known in the art which may be incorporated onto the surface of the device or otherwise integrated into the device. For example, well known radiopaque material include powdered tungsten, gold, iridium, platinum, barium, bismuth, iodine, iron and the like.

In various embodiments, the radiopaque materials may be incorporated over the entire device or in discrete regions in any number of patterns to allow for detection. In one embodiment, all or part of the balloon (60) may include radiopaque materials. In some embodiments the distal tip of the device includes radiopaque materials.

The device may conveniently be sized for passage through the lumen of an endoscope, laparoscope or other invasive scope device.

In another aspect, the present invention provides a method of treating a disease or condition of a subject. The method including delivering a fluid therapeutic to a surface of a tissue cavity of the subject using the device of the present invention.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The methodology and device of the present invention may be deployed to deliver a fluid to any body cavity. Generally, the device is deployed to a tissue cavity, such as a hollow organ of the body, for example the gastrointestinal tract or the bladder. As such, the term “body cavity” or “tissue cavity” is intended to refer to internal surfaces and spaces of the body. In exemplary embodiments, the device is deployed to the pulmonary or respiratory tract, gastrointestinal tract, or the pancreaticobiliary structure.

As used herein, the terms “condition,” “disease,” or “disorder” are used to refer to a variety of pathologies. For example, the term may include, but is not limited to, interstitial cystitis, various cancers, strictures, lesions and ulcers.

In one embodiment, the device is deployed to deliver a fluid to the bladder, for example to treat or ameliorate the symptoms of interstitial cystitis. Various fluids including medications may be instilled into the bladder via the device of the present invention.

In one embodiment, the medication dimethyl sulfoxide, or DMSO, (Rimso-50″) is placed into the bladder via a device of the present invention advanced through the urethra. Other medications that may be used include pentosan, hyaluronan, chondroitin, sodium bicarbonate, heparin and oxybutynin, either alone in any combination. The medication may further include an anesthetic, such as lidocaine or other anesthetic generally known to those ordinarily skilled in the art. In one embodiment the medication includes lidocaine, sodium bicarbonate and either pentosan or heparin to relieve urinary pain and urgency. In another embodiment the medication includes hyaluronan, chondroitin sulfate, oxybutynin, or any combination thereof.

After contacting the medication with the surface of the bladder for up to 5, 10, 15, 20, 25 minutes are longer, the medication is expelled through urination. Delivering medication directly to the bladder may reduce inflammation and possibly prevent muscle contractions that cause frequency, urgency and pain.

A typical treatment regime may include daily or weekly treatment for six to eight weeks, and subsequent maintenance treatments as needed, generally every one to two weeks, for up to a year or longer. As is generally appreciated by those of ordinary skill in the art, medications may affect liver function, so a physician may monitor liver function over time.

Although the invention has been described with reference to the above example, it will be understood that modifications and variations are encompassed within the spirit and scope of the invention. Accordingly, the invention is limited only by the following claims.

1. A catheter device for targeted delivery of a fluid, comprising:
a) a catheter defining at least one longitudinal lumen extending from a distal end of the catheter to a proximal end of the catheter; and
b) a deployable sponge being disposed within the lumen of the catheter in an un-deployed state, the sponge transitioning to an expanded state when advanced through a hole in the distal end of the catheter to deploy the sponge, thereby facilitating contact of the sponge with a tissue surface within a body cavity,
wherein the sponge is in fluid communication with the proximal end of the catheter via the at least one lumen to allow flow of a therapeutic fluid from the proximal end of the catheter to the sponge, and,
wherein further contact between the sponge and the tissue surface causes release of the therapeutic fluid from the sponge into the body cavity.

2. The device of claim 1, wherein the volume of the sponge when deployed is at least double as compared to the volume of the sponge when un-deployed.

3. The device of claim 1, wherein a portion of the sponge comprises a fluid resistant material such that the fluid may be targeted to a discrete tissue area.

4. The device of claim 1, wherein the device is configured for advancement though a lumen of an endoscope, a laparoscope, or an overtube.

5. The device of claim 1, further comprising an inflation balloon.

6. The device of claim 1, wherein the inflation balloon is disposed at the distal end of the catheter.

7. The device of claim 5, wherein the sponge is retrievable from deployment into the body cavity.

8. The device of claim 6, wherein the catheter further comprises an inflation lumen in fluid communication with the balloon whereby fluid or gas can be infused and withdrawn to inflate and to deflate the balloon, wherein further inflation of the balloon brings the sponge into contact with the tissue surface.

9. The device of claim 6, wherein the balloon further comprises a radiopaque material.

10. The device of claim 1, wherein the sponge is releasable from the catheter into the body cavity.

11. The device of claim 10, wherein the sponge is formed of bioresorbable material.

12. The device of claim 1, wherein the fluid is a medication-containing fluid.

13. A method for targeted delivery of a fluid to a tissue of a subject, comprising:
a) advancing the device according to any of claims 1 to 12 into a tissue cavity of the subject;
b) providing a fluid to the sponge via the at least one lumen; and
c) deploying the sponge through the hole in the distal end of the catheter thereby allowing contact of the sponge with a surface of the tissue cavity, wherein contact of the sponge with the surface of the tissue cavity allows delivery of the fluid to the surface.

14. The method of claim 13, wherein the device is advanced into the tissue cavity via a lumen of a catheter, an endoscope, a laparascope or an overtube.

15. The method of claim 13, wherein the tissue cavity is selected from gastrointestinal tract, bladder, pancreaticobiliary tree and peritoneum.

16. The method of claim 15, wherein the tissue cavity is the gastrointestinal tract, and peristaltic or catastaltic motion aids in the release of the therapeutic fluid from the sponge.

17. The method of claim 13, further comprising release of the deployed sponge into the tissue cavity.

18. The method of claim 17, further comprising retrieval of the deployed sponge from the tissue cavity.

19. The method of claim 13, wherein the device further comprises an inflation balloon comprising a radiopaque material, the balloon being inflated when the catheter is located at a desired location within the tissue cavity.

20. The method of claim 15, wherein the tissue cavity is the bladder.

21. The method of claim 20, wherein the device is advanced through the urethra into the bladder.

22-36. (canceled)
